(19) United States
(12) Patent Application Publication
(10) Pub. No.: US 2009/0226901 A1
(43) Pub. Date: Sep. 10, 2009

(54) Method of evaluating or selecting agents for preventing or improving pigmented spots

(75) Inventors: Daiki Murase, Tochigi (JP); Akira Hachiya, Cincinnati, OH (US); Yasuko Amano, Tochigi (JP)

Correspondence Address: STERNE, KESSLER, GOLDSTEIN & FOX P.L.L.C., 1100 NEW YORK AVENUE, N.W., WASHINGTON, DC, 20005, US

(73) Assignee: Kao Corporation

(21) Appl. No.: 12/073,568

(22) Filed: Mar. 6, 2008

(51) Int. Cl. C12Q 1/68; C12Q 1/02

(52) U.S. Cl. 435/6; 435/29

(57) ABSTRACT

The object of the present invention is to provide a method for evaluating or selecting an agent having preventive or alleviative effects on spots, which method realizes reliable and effective screening of candidate substances of the agent; a method for predicting spot formation; and a method for analyzing the condition of skin spots. The method of the present invention for evaluating or selecting an agent having preventive or alleviative effects on spots, includes evaluating a substance which suppresses a p53 activity, or an expression level of a p53 gene, a p53 target gene or an expression product thereof in an epidermal cell.

### TECHNICAL FIELD TO WHICH THE INVENTION PERTAINS

The present invention relates to a method for evaluating or selecting an agent having preventive or alleviative effects on spots.

### BACKGROUND ART

Spot is a kind of hyperpigmentation which progresses with aging, and are often formed on sun-exposed region of the skin. Spots are considered to be caused by long-term, repetitive exposure to sunlight. It has been reported that secretion of several cytokines which activates melanin synthesis in epidermal melanocytes is increased in epidermal keratinocytes at the lesional skin site (Non-Patent Documents 1 to 3).

Hitherto, various research findings have been reported on the mechanism of transient pigmentation (tanning) caused by UV rays (Non-Patent Document 4). On the other hand, the precise mechanism of chronic hyperpigmentations (e.g., senile lentigo), which are thought to be caused by irrespective of UV irradiation, has not yet been elucidated.

In recent years, the mechanism of chronic hyperpigmentation has been focused, and thus studies have been conducted on comprehensive gene expression analysis in senile lentigines (Non-Patent Document 5) or identification of associated factors employing human pigmented skin tissue (Patent Document 1).

It has been demonstrated several differences in the expression of some functional molecules between UV-induced pigmentation and chronic hyperpigmentation. For example, it has been reported that gene expression of IL-1α was increased by a single-dose of UV irradiation in human (Non-Patent Document 6), while decreased in the lesional skin of senile lentigo (Non-Patent Document 2).

In addition, expression pattern of the functional molecules have been found to differ between single-dose UV irradiation and continuous UV irradiation (Non-Patent Document 6). Therefore, it is strongly suggested that different mechanisms are involved in chronic pigmentation compared to transient pigmentation.

Meanwhile, hyperpigmentation such as senile lentigo is known to progress with aging, and may be generated at skin regions with non-excessively sun-exposure. Therefore, it has been pointed out that any factors other than UV irradiation may contribute to occurrence of senile lentigo (Non-Patent Document 7).

Thus, it has been indicated that chronic hyperpigmentations, such as senile lentigo, are associated with a mechanism different from that of transient pigmentation by UV irradiation.

The p53 gene is known as a tumor suppressor gene. The activation of p53 gene has been known to be involved in malignancy, prognosis or metastasis of many types of cancers, indicating that p53 plays an important role in suppression of malignant transformation.

In recent years, the pro-opiomelanocortin (POMC) gene has been newly identified as a transcription target of p53. It has been reported that p53 plays a prominent role in transient pigmentation caused by UV irradiation by activating transcription of the POMC gene, while not involved in basal pigmentation (Non-Patent Document 8). It has also been reported that several instances of UV-independent hyperpigmentation, observed in the treatment of several drugs with a DNA-damaging effect such as etoposide or 5-fluorouracil can be caused by UV-mimicking p53 activation, suggesting that p53 is also involved in UV-independent pigmentation (Non-Patent Document 8).

Furthermore, applying to epidermis an agent which activates p53 should be effective for the treatment or prevention of hyperproliferative, premalignant or UV-induced skin diseases (Patent Document 2).

However, there are no observations indicating the relationship between spots and an activity of p53, p53 gene, transcription targets of p53 gene (hereinafter referred to as p53 target gene), or their expression products.

Patent Document 1: JP3943490

Patent Document 2: JP1999-506755

Non-Patent Document 1: Saishin Hifukagaku Taikei Vol. 8, Dyschromia, Nakayama Shoten Co., Ltd.

Non-Patent Document 2: Kadono S., et al. (2001) J. Invest. Dermatol. 116: 571-577

Non-Patent Document 3: Hattori H., et al. (2004) J. Invest. Dermatol. 122: 1256-1265

Non-Patent Document 4: Enk CD., et al. (2004) Photodermatol. Photoimmunol. Photomed. 20: 129-137

Non-Patent Document 5: Aoki H., et al. (2007) Br. J. Dermatol. 156: 1214-1223

Non-Patent Document 6: Seite S., et al. (2004) Photochem. Photobiol. 79: 265-271

Non-Patent Document 7: Unver N., et al. (2006) Br. J. Dermatol. 155: 119-128

Non-Patent Document 8: Cui R., et al. (2007) Cell 128, 853-864

### SUMMARY OF THE INVENTION

Accordingly, the present invention provides the following.

1) A method for evaluating or selecting an agent having preventive or alleviative effects on spots, which includes evaluating a substance which suppresses an activity of p53, or an expression level of a p53 gene, a p53 target gene or an expression product thereof in an epidermal cell.

2) A method for evaluating or selecting an agent having preventive or alleviative effects on spots, which includes the following steps (1) to (4):

(1) a step of contacting a test substance to an epidermal cell, wherein the epidermal cell has an activity of p53, or a p53 gene, a p53 target gene or an expression products thereof expressed therein;

(2) a step of measuring the activity of p53, or the expression level of the p53 gene, the p53 target gene or the expression product thereof in the epidermal cell;

(3) a step of comparing the activity or the expression level measured in step (2) with an activity of p53, or an expression level of a p53 gene, a p53 target gene or an expression product thereof in a control epidermal cell which has not been contacted to the test substance; and

(4) a step of selecting, on the basis of the results obtained in step (3), the test substance that has suppressed the activity of p53, or the expression level of the p53 gene, the p53 target gene or the expression product thereof, as an agent having preventive or alleviative effects on spots.

3) A method for evaluating or selecting an agent for regulating expression of SCF or endothelin-1, which includes evaluating a substance which suppresses an activity of p53, or an expression level of a p53 gene or a p53 target gene or an expression product thereof in an epidermal cell.

4) A method for analyzing a condition of skin spots in human subject, which includes determining the degree of progression or alleviation of formation of skin spots on the basis of an activity of p53, or an expression level of a p53 gene or a p53 target gene or an expression product thereof in a human epidermal cell.

### BRIEF DESCRIPTION OF THE DRAWINGS

FIG. 1 shows the expression of p53 in human skin tissue.

FIG. 2 shows the expression levels of CDKN1A (p21) gene, GADD45A gene and MDM2 gene in human epidermal tissue.

FIG. 3 shows changes in the expression level of SCF (KITLG) gene and endothelin-1 gene by 5-fluorouracil (5-FU) treatment in cultured epidermal keratinocytes.

FIG. 4 shows changes in the expression level of SCF (KITLG) gene and endothelin-1 gene by pifithrin-α (PFT) in cultured epidermal keratinocytes.

FIG. 5 is a graph showing changes in melanin contents of a three-dimensional cultured skin substitute with or without pifithrin-α (PFT) treatment.

FIG. 6 shows changes in expression level of tyrosinase gene, SCF (KITLG) gene and endothelin-1 gene in organ-cultured skin tissue of pigmentatio petaloides actinica with or without pifithrin-α (PFT) treatment.

### DETAILED DESCRIPTION OF THE INVENTION

The present invention provides a method for evaluating or selecting an agent having preventive or alleviative effects on spots. The method can realize reliable and effective screening of candidate substances of the agent. The present invention also provides a method for analyzing the conditions of spots.

The present inventors have conducted studies on the detailed mechanisms of spot formation in molecular levels, and have found that the expression level of a p53 gene, a p53 target gene or an expression product thereof is considerably increased in epidermal cells (keratinocytes) of skin tissue on which spots have been formed, and thereby found that the activity of p53, or the expression level of the p53 gene, the p53 target gene or the expression product thereof can be employed as an index for evaluating or selecting an agent having preventive or alleviating effects on spots, or for analyzing the condition of spots.

The present invention can realize reliable and effective evaluation or selection of an agent having preventive or alleviating effects on spots. The present invention can also realize objective evaluation of the degree of progression or alleviation of formation of spots.

As used herein, the term “spots” generally is referred to as senile lentigines, but also encompasses brown or dark brown pigmented sites which appear on the skin due to chronically deposited or remaining pigments such as melanin; for example, pigmentatio petaloides actinica, ephelides, freckles and chloasma.

The “p53 gene,” which is known as a tumor suppressor gene, is a gene most frequently mutated in cancer. An expression product of the p53 gene functions as a transcription factor to activate transcription of genes involved in, for example, cell cycle regulation, apoptosis induction, DNA repair, and cellular senescence. It has been known that abnormality in the p53 gene causes reduction in the amount (inactivation) of p53 target genes, resulting in malignant transformation of cells.

There are many p53 target genes. Examples thereof include GADD45A gene, which is involved in DNA repair and cell cycle regulation; p53R2 gene and XPC gene, which are involved in DNA repair; CDKN1A (p21) gene, 14-3-3δ gene, and cdc25C gene, which are involved in cell cycle regulation; MDM2 gene and cyclin G gene, which are involved in p53 functional regulation; and BAX gene, PUMA gene, and PIG3 gene, which are involved in apoptosis.

Each of the methods of the present invention may employ an expression product of any genes as described above, such as p53 protein, Gadd45a protein, p53R2 protein, XPC protein, p21 protein, Mdm2 protein and Bax protein.

As described in the Examples herein below, when the expression of p53 protein was analyzed for a skin tissue collected from a skin region with human senile lentigines, virtually no p53 expression was observed in a site in the vicinity of the site of senile lentigines, whereas a considerable increase in p53 expression was observed in the epidermis from the site of senile lentigines. In addition, when the expression of p53 target genes was analyzed in the epidermis of healthy male subjects collected from the skin at a site of spots, a site in the vicinity of the site of spots (outside of forearm) or sun-protected areas (inside of upper arm), the expression levels of p53 target genes such as CDKN1A (p21) gene, GADD45A gene and MDM2 gene were found to be significantly increased in spots compared to vicinity and sun-protected areas.

When gene expression analysis was performed in cultured human epidermal keratinocytes with the treatment with 5-fluorouracil, which is a known p53 activator, the expression levels of SCF (KITLG) gene and endothelin-1 gene were found to be significantly increased, whereas when cells were treated with pifithrin-α (PFT), a known p53 inhibitor, the expression levels of SCF (KITLG) gene and endothelin-1 gene were found to be significantly decreased.

When PFT was added to a three-dimensional cultured skin substitute constituted by normal human epidermal cells (keratinocytes) and pigment cells (melanocytes), the melanin contents in the skin substitute was decreased in a dose-dependent manner with PFT treatment, whereas when PFT was added to organ-cultured tissues of human pigmentatio petaloides actinica, the expression levels of tyrosinase gene and SCF (KITLG) gene, which play an important role in melanin synthesis, were significantly decreased. Similarly, expression of endothelin-1 gene tended to be significantly decreased. SCF (stem cell factor) is a ligand for the c-kit receptor expressed on the surface of some kinds of cells such as hematopoietic stem cells, and is known as a membrane-bound growth factor which promotes growth and differentiation of hematopoietic cells. In recent years, c-kit has been elucidated to be expressed not only on the surface of hematopoietic cells, but also on the surface of mast cells, melanocytes or germ cells (J. Exp. Med., 183, 2681-2686, 1996). As has also been recently reported, in transgenic mice expressing SCF only in the skin, melanin synthesis was enhanced through induction of mast cells and growth of melanocytes (J. Exp. Med., 187, 1565-1573, 1998). Therefore, SCF is considered to be strongly involved in overproduction of melanin in the skin.

Comprehensive research findings on p53 transcriptional target genes in the human genome were reported in 2006 (Cell, 124, 207-219, 2006). The report indicates that SCF (KITLG) gene was identified as a new p53 target gene, and expression level of the gene was induced by the addition of 5-fluorouracil to HCT116 cells (i.e., a human colon cancer cell line). However, the relationship between p53 and SCF in the skin or epidermal tissue has not yet been elucidated.

Endothelin-1 has been known as a cytokine whose production from epidermal keratinocytes is increased after UVB irradiation, and has been indicated to be deeply involved in overproduction of melanin in epidermis (Pigment Cell Res., 10, 218-228, 1997).

It has been shown that the aforementioned cytokines are overexpressed at the lesion of chronic hyperpigmentation, and these cytokines synergistically promotes pigmentation (Am. J. Pathol., 165, 2099-2109, 2004).

These findings indicate that p53 activity, or the expression of the p53 gene, a p53 target gene or an expression product thereof in the epidermal cells is deeply involved in an upstream mechanism of chronic hyperpigmentation contributing to formation of spots. Therefore, these data suggest that a substance which suppresses the activity or expression of p53 is useful as an agent having preventive or alleviative effects on spots, or as an agent for regulating expression of SCF or endothelin-1, and that an agent having preventive or alleviative effects on spots, or an agent for regulating expression of SCF or endothelin-1 can be evaluated or selected on the basis of the p53 activity, or of the expression level of the p53 gene, the p53 target gene or the expression product thereof. The condition of spots in the skin (e.g., the degree of progression or alleviation of formation of spots) can be analyzed on the basis of the p53 activity, or the expression level of the p53 gene, the p53 target gene or the expression product thereof.

### 1) Method for Evaluating or Selecting an Agent Having Preventive or Alleviative Effects on Spots

This method selects, as a candidate, a substance which suppresses the p53 activity, or the expression level of the p53 gene, a p53 target gene or an expression product thereof in an epidermal cell.

No particular limitation is imposed on the epidermal cell employed, so long as it is derived from epidermal cells of animals having p53 activity, or the p53 gene, a p53 target gene or an expression product thereof expressed therein. Preferably, the expression level of the gene or the protein in the epidermal cell is increased compared to a normal epidermal cell. The epidermal cell employed may be included in or isolated from the skin tissue, or may be derived from normal epidermal cells or cells of an established epidermal cell line (e.g., HaCaT cells).

The animal from which epidermal cells are derived may be a nonhuman animal (e.g., a rodent such as mouse, rat or guinea pig), but is preferably a human.

The tissue from which epidermal cells are derived may be, for example, skin tissue surgically excised from a living organism; skin tissue surgically excised from a living organism and implanted into another animal such as an immunodeficient mouse; a cultured skin substitute constituted by epidermal cells and other skin-constitutive cells; or skin reconstituted from immunodeficient mice or other animals.

As used herein, “p53 activity” is referred to as, for example, an ability of p53 protein for activating transcription of a target gene, or an ability for protein modification such as phosphorylation or acetylation which indicating the activity level of the protein. “Substance which suppresses p53 activity” as used herein is referred to as a substance which regulates, for example, the ability for activating transcription or protein modification as described above.

As used herein, “substance which suppresses an expression level of the p53 gene or a p53 target gene” is referred to as a substance which suppresses expression of mRNA complementary to a polynucleotide constituting the p53 gene or a p53 target gene, or promotes degradation of the mRNA; and “substance which suppresses an expression level of an expression product thereof” is referred to as a substance which suppresses the levels of the expression product of the p53 gene or the p53 target gene (e.g., p53 protein), or promotes degradation of the expression product to thereby suppress the expression level.

In the evaluating or selecting method of the present invention, selection of a candidate substance on the basis of the p53 gene or the p53 target gene is specifically carried out through, for example, the following steps:

(1a) a step of contacting a test substance to an epidermal cell having the p53 gene or a p53 target gene expressed therein;

(2a) a step of measuring the expression level of the p53 gene or the p53 target gene in the epidermal cell to which the test substance has been contacted;

(3a) a step of comparing the expression level measured in step (2a) with the expression level of corresponding gene in a control epidermal cell which has not been contacted to the test substance; and

(4a) a step of selecting, on the basis of the results obtained in step (3a), the test substance that has suppressed the expression level of the p53 gene or the p53 target gene, as an agent having preventive or alleviative effects on spots.

In step (1a), contact of the test substance to the epidermal cell may be carried out by dissolving the test substance in a solvent (e.g., ethanol, DMSO, or water) to prepare a test substance solution (0.0001 to 10 w/v %), followed by applying the solution (which is employed as is or appropriately diluted before use) to the epidermal cell or to a medium culturing the epidermal cell.

Detection or quantitative determination of gene expression may be carried out through a known method such as RT-PCR, by use of RNA prepared from the aforementioned tissue, or a complementary polynucleotide transcribed from the RNA.

In the evaluating or selecting method of the present invention, selection of a candidate substance on the basis of the expression level of the expression product of the p53 gene or a p53 target gene is specifically carried out through, for example, the following steps:

(1b) a step of contacting a test substance to an epidermal cell having the expression product of the p53 gene or a p53 target gene expressed therein;

(2b) a step of measuring the expression level of the expression product in the epidermal cell;

(3b) a step of comparing the expression level measured in step (2b) with the expression level of the corresponding gene expression product in a control epidermal cell which has not been contacted to the test substance; and

(4b) a step of selecting, on the basis of the results obtained in step (3b), the test substance that has suppressed the expression level of the expression product, as an agent having preventive or alleviative effects on spots.

In step (1b), contact of the test substance to the epidermal cells may be carried out in a manner similar to that described above in step (1a).

In step (2b), the expression level of the expression product may be quantitatively determined through a known technique such as western blotting by use of an antibody which recognizes the expression product (e.g., anti-p53 antibody, anti-p21 antibody, anti-Gadd45a antibody, or anti-Mdm2 antibody).

Western blotting can be carried out with a primary antibody which recognizes the expression product and a secondary antibody which binds to the primary antibody and is labeled with a radioisotope (e.g., ¹²⁵I), a fluorescent substance or an enzyme (e.g., horseradish peroxidase (HRP)), by measuring a signal from the labeling substance by means of a radiation meter, a fluorescence detector and the like.

The antibody may be a monoclonal antibody or a polyclonal antibody prepared by using the expression product as an immunogen. The antibody may be a commercially available one which is guaranteed to recognize the expression product, or may be prepared through immunization of an animal (e.g., rabbit or mouse).

In the evaluating or selecting method of the present invention, selection of a candidate substance on the basis of the p53 activity is specifically carried out through, for example, the following steps:

(1c) a step of contacting a test substance to an epidermal cell having p53 activity;

(2c) a step of measuring the p53 activity in the epidermal cell;

(3c) a step of comparing the p53 activity measured in step (2c) with the p53 activity in a control epidermal cell which has not been contacted to the test substance; and

(4c) a step of selecting, on the basis of the results obtained in step (3c), the test substance that has suppressed p53 activity, as an agent having preventive or alleviative effects on spots.

In step (1c), contact of the test substance to the epidermal cell may be carried out in a manner similar to that described above in step (1a).

In step (2c), p53 activity may be quantitatively determined through a known technique such as western blotting by use of an antibody which recognizes a modification of p53 protein which reflects the activation of the p53 (e.g., an anti-phosphorylated p53 antibody or an anti-acetylated p53 antibody). One or more modification (phosphorylation or acetylation) sites in p53 protein may be measured, and various kinds of modification sites, which have been identified as p53 modification sites, may be detected.

Western blotting can be carried out with a primary antibody which recognizes a modification of p53 protein which reflects the activation of p53 and a secondary antibody which binds to the primary antibody and is labeled with a radioisotope (e.g., ¹²⁵I), a fluorescent substance or an enzyme (e.g., horseradish peroxidase (HRP)), by measuring a signal from the labeling substance by means of a radiation meter, a fluorescence detector and the like.

The antibody may be a monoclonal antibody or a polyclonal antibody prepared by using the aforementioned modified protein as an immunogen. The antibody may be a commercially available one, which is guaranteed to recognize the aforementioned modification site, or may be prepared through immunization of an animal (e.g., rabbit or mouse).

In step (2c), quantitative determination of p53 activity may be carried out through another known technique, for example, the gel shift assay using a cell extract from the epidermal cell having p53 activity and a DNA fragment containing a sequence which binds specifically to p53 and being labeled with, for example, a radioisotope (e.g., ³²P) or a fluorescent substance.

The gel shift assay can be carried out through the following procedure: a cell extract from the epidermal cell having p53 activity is mixed with the DNA fragment containing a sequence which binds specifically to p53 and being labeled with, for example, a radioisotope (e.g., ³²P) or a fluorescent substance; the mixture is incubated and subjected to polyacrylamide gel electrophoresis; and a signal from the labeling substance is measured by means of a radiation meter, a fluorescence detector and the like.

The sequence which binds specifically to p53 may be obtained from literature or existing databases. The DNA fragment may be prepared through a conventionally known technique.

In step (2c), quantitative determination of p53 activity may be carried out through another known technique; for example, a reporter gene assay in which a reporter gene (plasmid) having an expression promoter containing a sequence which binds specifically to p53 is introduced into the epidermal cell having p53 activity.

The reporter gene assay can be carried out through the following procedure: a reporter gene (plasmid) which incorporating a gene expressing an enzyme such as firefly luciferase and an expression promoter containing a sequence which binds specifically to p53 is introduced into the epidermal cell having p53 activity; and the intensity of fluorescence or color development by enzymatic reaction is measured by means of a detector.

The reporter gene (plasmid) may be prepared through a conventionally known technique.

As described above, on the basis of the quantitatively determined p53 activity, or the quantitatively determined expression level of the p53 gene, a p53 target gene or an expression product thereof, the test substance can be selected as a substance which suppresses the p53 activity, or expression of the p53 gene, the p53 target gene or the p53 protein. Specifically, when the thus-determined p53 activity or expression level is statistically significantly suppressed relative to the corresponding activity or expression level in a control epidermal cell, the test substance is selected as a substance which suppresses p53 activity or p53 expression.

### 2) Method for Analyzing the Condition of Spot

According to the method for analyzing the condition of spots of the present invention, the condition of spots can be analyzed by, for example, determining whether or not p53 activity, or the expression level of the p53 gene, the a p53 target gene or an expression product thereof in an epidermal cell at a site where spots are observed is higher than the corresponding activity or expression level in a normal epidermal cell. A normal skin site employed as a control may be a site in the vicinity of the site of spots, or may be a site protected to sunlight and may be a site having relatively few spots.

Specifically, when p53 activity, or the expression level of the p53 gene, the p53 target gene or the expression product thereof in the epidermal cell at a skin site of spots is higher than the corresponding activity or expression level in an epidermal cell at a normal site (control), the degree of progression or alleviation of formation of spots on the skin site can be determined, and thereby the condition of skin spots can be analyzed.

The test sample employed for analysis may be skin tissue collected through a skin biopsy (e.g., punch biopsy), or may be epidermal tissue collected through, for example, the suction blister method. The test sample may be epidermal tissue isolated from the collected skin tissue through, for example, enzymatic treatment or thermal treatment, or may be derived from an organ culture of the collected skin or epidermal tissue. Alternatively, the test sample may be an epidermal cell isolated from the skin or epidermal tissue, or may be an appropriately cultured epidermal cell.

### EXAMPLES

### Example 1

In the present Example, p53 expression in human skin tissue was analyzed through immunohistochemistry.

Skin tissue was collected from human skins with senile lentigines, and paraffin sections of the tissue were prepared. After removal of paraffin, each of the tissue sections was thermally treated in REAL™ Target Retrieval Solution (product of DAKO) at 95° C. for 45 minutes to activate antigens. After cooling at ambient temperature for 30 minutes, the tissue section was washed with phosphate buffered saline (PBS), and then treated with 0.3% H₂O₂ solution for 30 minutes. After washing with PBS, the tissue section was blocked with 10% normal goat serum (product of Nichirei Bioscience) at room temperature for one hour, and then mouse anti-human p53 antibody (DO-7, product of DAKO) 100-fold diluted with a blocking solution (primary antibody) was added to the tissue section, followed by allowing the section to stand still at 4° C. overnight. After washing with PBS, peroxidase-labeled anti-mouse IgG polyclonal antibody (Fab′) (product of Nichirei Bioscience) (secondary antibody) was added to the tissue section, followed by allowing the section to stand still at room temperature for 30 minutes. After washing with PBS, the tissue section was subjected to enzymatic reaction by use of HistoMark® TrueBlue™ Peroxidase System (product of KPL). After observing color development, water was added to terminate the reaction, and then the tissue section was subjected to counterstaining with NUCLEAR FAST RED (product of Vector Laboratories). After washing with running water for 10 minutes, the thus-stained tissue section was sealed with glycerin and a coverslip, and a stained tissue image was obtained by means of a microscope and an imaging apparatus (product of Carl Zeiss).

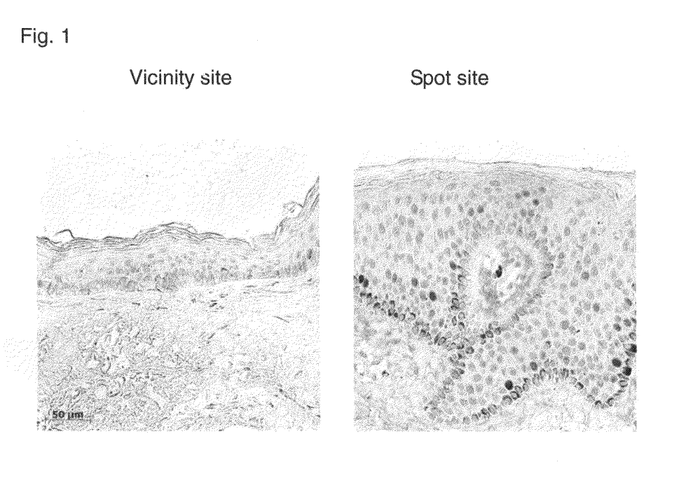

As shown in FIG. 1, virtually no p53 expression was observed at a site in the vicinity of the site of senile lentigines, whereas a considerable increase in expression level of p53 was observed in epidermis from the site of senile lentigines (blue-stained site).

### Example 2

In the present Example, expression of p53 target genes in human epidermal tissue was analyzed.

Firstly, epidermis was obtained, through the suction blister method, from the skin of healthy male subjects at a site of spots, a site in the vicinity of the site of spots (outside of forearm), or a site in the sun-protected area (inside of upper arm). Specifically, each of the test sites was sterilized, and a 1.0- to 2.5-mm-diameter syringe (product of Terumo Corporation) was brought into contact with a skin surface, followed by suction by means of a pump for about one to about two hours, to thereby separate epidermis from dermis. The thus-separated epidermis was isolated with sterilized scissors to prepare a sample derived from the subject.

The prepared sample of epidermal tissue was washed with PBS, and then transferred into a tube containing RNAlater™ (product of QIAGEN) (1 mL), followed by allowing the tube to stand still at 4° C. overnight. Subsequently, the tissue sample was washed with PBS, and total RNA was prepared through a customary method by use of RNeasy micro kit (product of QIAGEN).

An aliquot (1 μg) of the resultant RNA solution was subjected to reverse transcription for synthesis of cDNA. Reverse transcription was carried out through a customary method using SuperScript III First-Strand Synthesis System for RT-PCR (product of Invitrogen Corporation) and Peltier Thermal Cycler (product of MJ Research, Inc.).

The thus-synthesized cDNA was employed for gene expression analysis through quantitative PCR using TaqMan probe. TaqMan® Gene Expresson Assays (P/N 4331182) (product of Applied Biosystems) was employed for probes and primers specific to p53 target genes. The expression level of each of the genes was directly quantified through the calibration curve method and was corrected by the expression level of internal control standard gene RPLP0 (Assay ID: Hs99999902_m1). Reaction was carried out under a customary condition by means of a sequence detector (ABI PRISM 7500 Real Time PCR System, product of Applied Biosystems).

Specifically, in the present Example, the expression levels of CDKNlA (p21) gene (Assay ID: Hs00355782_ml), GADD45A gene (Assay ID: Hs00169255_ml) and MDM2 gene (Assay ID: Hs01066938_ml) were measured. These genes are conventionally known p53 target genes.

Probes and primers employed for measuring the expression levels of these genes may be commercially available products designed for gene expression quantification. Alternatively, probes and primers specific to target genes may be designed and prepared. Specifically, such gene-specific probes and primers may be designed and employed with a design software (e.g., Primer Express (product of Applied Biosystems)) on the basis of gene sequence data (e.g., cDNA) obtained from databases.

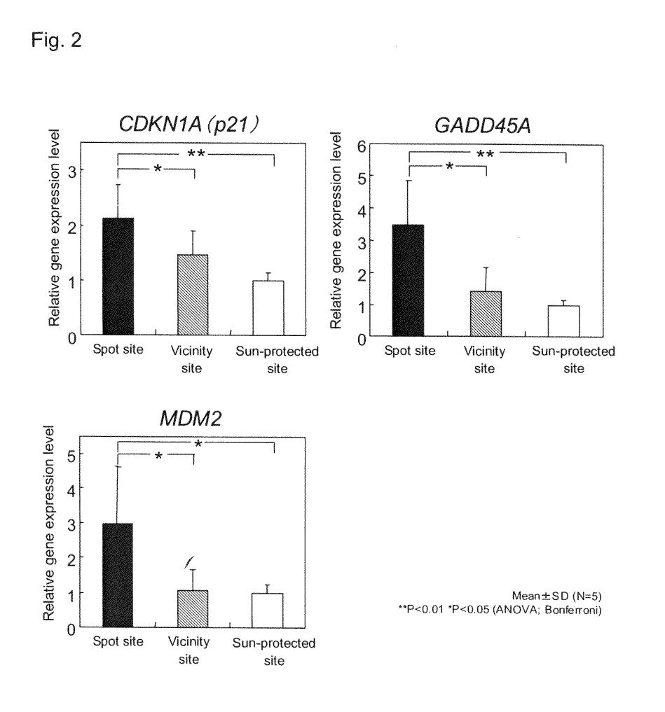

As shown in FIG. 2, expression of CDKN1A (p21) gene, GADD45A gene or MDM2 gene, which are known p53 target genes, was significantly increased at the site of spots.

### Example 3

Human epidermal cells derived from neonatal foreskin were purchased from Kurabo Industries Ltd., and were precultured in a serum-free basal growth medium (EpiLife, product of Kurabo Industries Ltd.) in an atmosphere of 5% (v/v) CO₂ at 37° C. The thus-precultured epidermal cells were inoculated into a 6-well culture plate (product of Falcon) (1.0×10⁵ cells/well). Twenty-four hours thereafter, the cells were cultured in a growth medium without including BPE (bovine pituitary gland extract) and hEGF (human epidermal growth factor), and 24 hours thereafter, 5-fluorouracil (5-FU, product of Calbiochem), which is a conventionally known p53 activator, was added at a concentration of 1 or 10 μg/mL. After 48-hour treatment, total RNA was extracted by use of TRIzol® Reagent (product of Invitrogen Corporation). Specifically, each well of the culture plate was washed with PBS, and TRIzol® Reagent (1 mL) was added to the well. The resultant mixture was recovered in a 1.5-mL tube, Chloroform (200 μL) was added thereto, and the mixture was thoroughly stirred, followed by centrifugation at 15,000 rpm for 15 minutes. The resultant upper layer was transferred into a fresh 1.5-mL tube, and an equiamount of isopropanol was added thereto, followed by stirring and then centrifugation at 12,000 rpm for 10 minutes. The supernatant was aspirated, and 75% ethanol (1 mL) was added to the precipitate, followed by centrifugation at 8,500 rpm for five minutes. The supernatant was removed through aspiration, and then the precipitate was dissolved in dH₂O (15 to 30 μL), to thereby prepare total RNA.

The thus-extracted total RNA was subjected to reverse transcription to synthesize cDNA, and the thus-synthesized cDNA was employed for gene expression analysis through quantitative PCR using TaqMan® probe. In the present Example, specifically, expression levels of SCF (KITLG) gene (Assay ID: Hs00241497_m1) and endothelin-1 gene (Assay ID: Hs00174961_m1) were measured. Detail procedure for gene expression analysis used in the present Example was similar to that used in Example 2.

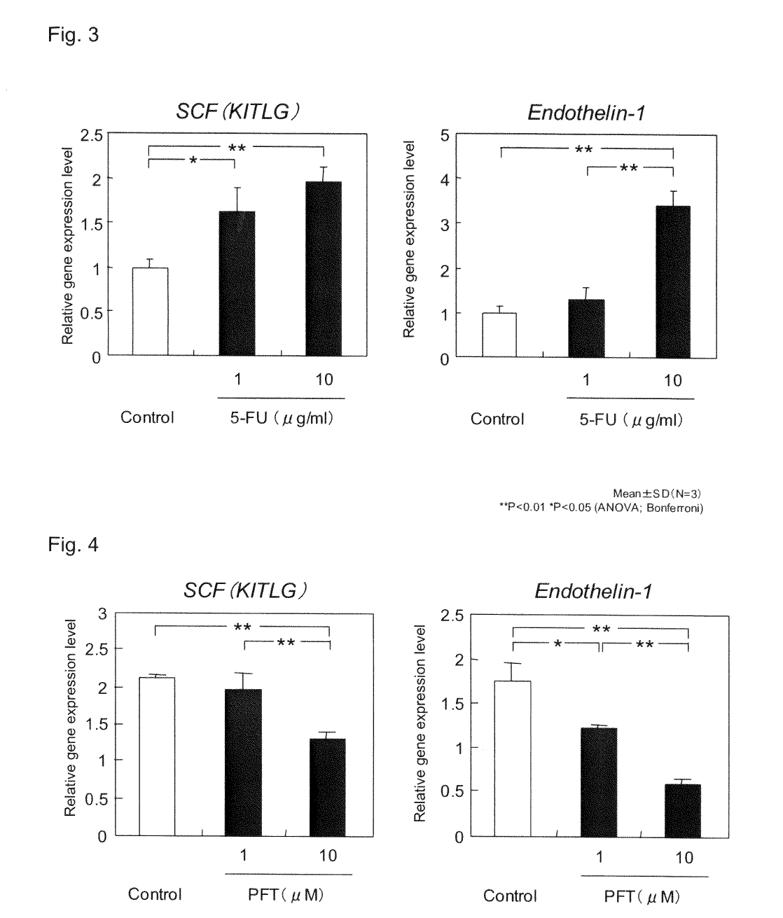

As shown in FIG. 3, expression of SCF (KITLG) gene or endothelin-1 gene in cultured epidermal cells was significantly induced by 5-fluorouracil (5-FU), which is a conventionally known p53 activator.

### Example 4

Human epidermal cells derived from neonatal foreskin were purchased from Kurabo Industries Ltd., and were precultured in a serum-free basal growth medium (EpiLife, product of Kurabo Industries Ltd.) in an atmosphere of 5% (v/v) CO₂ at 37° C. The thus-precultured epidermal cells were inoculated into a 6-well culture plate (product of Falcon) (1.0×10⁵ cells/well). Twenty-four hours thereafter, the cells were cultured in a growth medium without including BPE (bovine pituitary gland extract) and hEGF (human epidermal growth factor), and 24 hours thereafter, pifithrin-α (PFT, product of Calbiochem), which is a conventionally known p53 inhibitor, was added at a concentration of 1 or 10 μM. After 24-hours culturing, total RNA was extracted by use of TRIzol® Reagent (product of Invitrogen Corporation). The thus-extracted total RNA was subjected to reverse transcription to synthesize cDNA, and the thus-synthesized cDNA was employed for gene expression analysis through quantitative PCR using TaqMan® probe. In the present Example, specifically, expression levels of SCF (KITLG) gene and endothelin-1 gene were measured. Detail procedure for gene expression analysis used in the present Example was similar to that used in Example 2 or 3.

As shown in FIG. 4, expression of SCF (KITLG) gene or endothelin-1 gene in cultured epidermal cells was significantly decreased by pifithrin-α (PFT), which is a conventionally known p53 inhibitor.

### Example 5

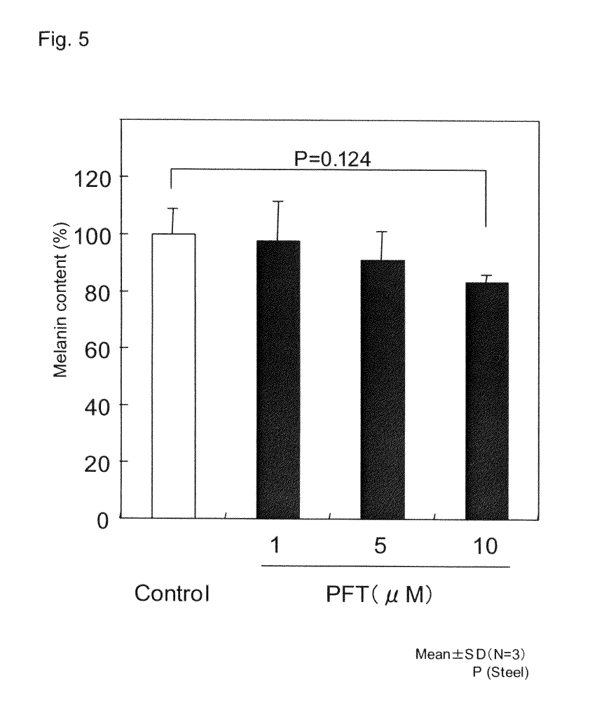

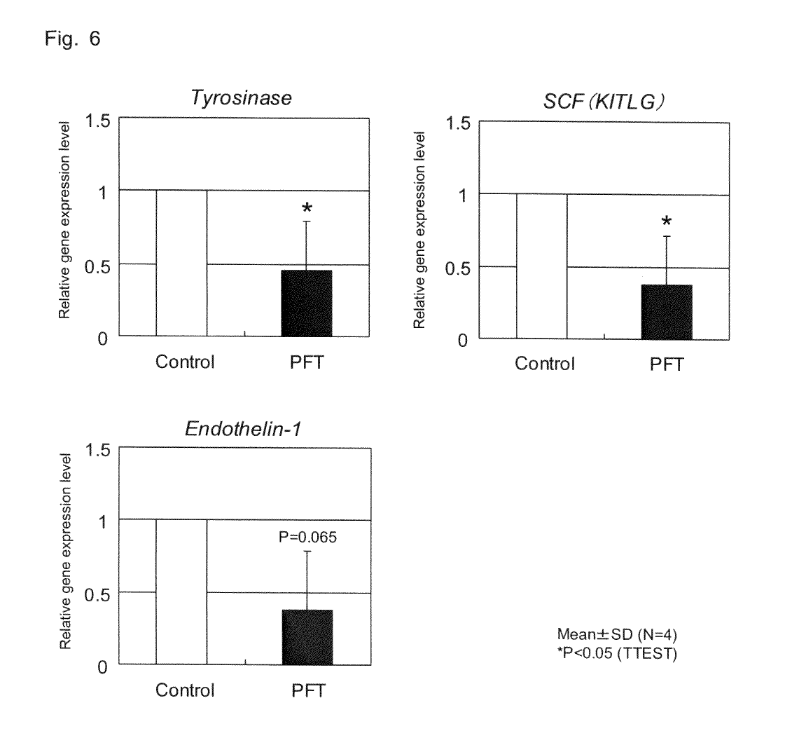

A three-dimensional cultured skin model (MEL-300A) was purchased from Kurabo Industries Ltd. Immediately after the product had been obtained, a tissue cup was transferred into a 6-well culture plate (product of Falcon), and cultured overnight in a long-term maintenance medium (EPI-100-NMM-113, product of Kurabo Industries Ltd.) (1 mL), in an atmosphere of 56 (v/v) CO₂ at 37° C. Subsequently, pifithrin-α (PFT), which is a conventionally known p53 inhibitor, was added, followed by culturing for 14 days. Culturing was carried out by allowing the tissue cup to stand still on a sterilized stainless steel washer (product of Kurabo Industries Ltd.) and by use of a long-term maintenance medium (5 mL). Medium was exchanged every three days. After culturing, skin tissue was removed from the tissue cup with tweezers, and then washed thrice with PBS, once with 5% (v/v) trichloroacetic acid, and once with a mixture of diethyl ether and ethanol (1:3 by volume). Thereafter, the thus-washed skin tissue was incubated at 50° C. for two hours, to thereby dry the tissue. Subsequently, the tissue was dissolved in 2N aqueous sodium hydroxide solution (2N) (200 μL), followed by centrifugation at 15,000 rpm for 15 minutes. Thereafter, the culture supernatant was recovered, and absorbance was measured at 405 nm, to thereby calculate the melanin content in the tissue. FIG. 5 shows relative contents of melanin to control (100%).

As shown in FIG. 5, pifithrin-α (PFT) (i.e., a conventionally known p53 inhibitor) decreased the melanin contents of the three-dimensional cultured skin model in a dose-dependent manner of PFT.

### Example 6

Through a contract laboratory of Stephens & Associates (Carrollton, Tex.), subjects having spots were recruited. A dermatologist, contractor of the laboratory, collected samples of skin tissue with pigmentatio petaloides actinica from the shoulder (at two sites for each subject) in two 56-year-old Caucasian female subjects at with a biopsy trepan (diameter: 4 mm). Thereafter, the thus-collected skin biopsy samples were immersed in Dulbecco's modified Eagle's medium (DMEM), and transported to the Cincinnati branch of Biological Science Americas Laboratories while being maintained at about 4 to 10° C. Within 24 hours after collection of the samples of skin tissue with pigmentatio petaloides actinica, each of the samples was cut into halves with a disposable scalpel. One half of the sample was organ-cultured in DMEM containing 10 μM pifithrin-α (PFT) (a p53 inhibitor), and the other half was organ-cultured in DMEM containing no pifithrin-α (PFT). A collagen sponge (Avitene Ultrafoam, MedChem Products, Inc. Woburn, Mass.) was cut so as to be received in the inner well an organ culture dish (BD Biosciences (San Jose, Calif.)), and to make a hole (diameter: 3 mm) in the center of the sponge. Organ culture was initiated after the pigmentatio petaloides actinica tissue sample had been placed in the hole so that the surface of the sample was flush with the sponge surface. The outer well of the organ culture dish was charged with PBS (5 mL), and organ culture was initiated in an incubator at 5% CO₂ and 37° C. Medium was exchanged 24 hours and 48 hours after initiation of organ culture. Seventy-two hours after initiation of organ culture, the pigmentatio petaloides actinica tissue sample was treated with hot water of 70° C. for one minute, and only epidermis was recovered in RNAlater (Qiagen, Valencia, Calif.).

Thereafter, total RNA was extracted by use of RNeasy micro kit (Qiagen), and cDNA was synthesized through a customary method by means of ThermoScript RT-PCR Systems (Invitrogen, Carlsbad, Calif.). Probes specific to tyrosinase mRNA, SCF (KITLG) mRNA, and endothelin-1 mRNA were obtained from TaqMan® Gene Expression Assays (Applied Biosystems) (Assay IDs: Hs00165976_m1, Hs00241497_m1, and Hs00174961_ml), and real-time quantitative RT-PCR was carried out by means of ABI PRISM 7300 sequence detection system (Applied Biosystems). The expression level of each gene was corrected by the expression level of internal control standard gene RPLPO (Assay ID: Hs99999902_ml). The expression level of each gene in the PFT-treated spot sample was calculated relative to that of the gene in a control spot sample (taken as 1).

As shown in FIG. 6, in association with inhibition of p53, the expression of tyrosinase gene or SCF (KITLG) gene was significantly decreased, and the expression of endothelin-1 gene tended to be significantly decreased.

1. A method for evaluating or selecting an agent having preventive or alleviative effects on spots, comprising evaluating a substance which suppresses an activity of p53, or an expression level of a p53 gene, a p53 target gene or an expression product thereof in an epidermal cell.

2. A method for evaluating or selecting an agent having preventive or alleviative effects on spots, comprising the following steps (1) to (4):
(1) a step of contacting a test substance to an epidermal cell, wherein the epidermal cell has an activity of p53, or a p53 gene, a p53 target gene or an expression product thereof expressed therein;
(2) a step of measuring the activity of p53, or the expression level of the p53 gene, the p53 target gene or the expression product thereof in the epidermal cell;
(3) a step of comparing the activity or the expression level measured in step (2) with an activity of p53, or an expression level of a p53 gene, a p53 target gene or an expression product thereof in a control epidermal cell which has not been contacted to the test substance; and
(4) a step of selecting, on the basis of the results obtained in step (3), the test substance that has suppressed the activity of p53, or the expression level of the p53 gene, the p53 target gene or the expression product thereof, as an agent having preventive or alleviative effects on spots.

3. The method as described in claim 1 or 2, wherein the p53 target gene is at least one selected from GADD45A gene, CDKN1A (p21) gene and MDM2 gene.

4. A method for evaluating or selecting an agent for regulating expression of SCF or endothelin-1, comprising evaluating a substance which suppresses an activity of p53, or an expression level of a p53 gene, a p53 target gene or an expression product thereof in an epidermal cell.

5. A method for analyzing the condition of skin spots, comprising determining the degree of progression or alleviation of formation of skin spots by comparing an activity of p53, or an expression level of a p53 gene, a p53 target gene or an expression product thereof in a human epidermal cell with an activity of p53, or an expression level of a p53 gene, a p53 target gene or an expression product thereof in a control epidermal cell.
